(19) United States
(12) Patent Application Publication
(10) Pub. No.: US 2007/0203504 A1
(43) Pub. Date: Aug. 30, 2007

(54) Skin-marking devices and their use

(76) Inventors: Blair Denny, Springdale, MD (US); Rebecca Moreland, District Heights, MD (US)

Correspondence Address: BIOTECHPAATENT.NET LLC, No 221, 5284 Randolph Road, Rockville, MD, 20852, US

(21) Appl. No.: 11/361,997

(22) Filed: Feb. 27, 2006

(51) Int. Cl. A61B 17/00

(52) U.S. Cl. 606/116

(57) ABSTRACT

An anti-microbial growth-inhibiting, skin-marking device for applying a marking agent to the skin of a patient who is to undergo a medical procedure such as radiation therapy. The skin-marking device has a flexible housing that contains a rupturable cartridge containing the marking agent and anti-bacterial, anti-fungal, and anti-viral solution. A flexible housing with a dry wick-type marking nib in one end which communicates with the interior of the housing and which is impregnated with a skin marking dye or ink. A cap for the marking device which is resistant to mocrobials, mold and mildew. The application end is used to tattoo, or paint or mark the skin of the patient, without penetrating the surface of the skin, applying the marking agent to mark treatment set-up points.

### FIELD OF THE INVENTION

This invention relates generally to a medical marking device, and specifically to a novel anti-bacterial, anti-viral, and anti-fungal skin-marking device adapted for marking or tattooing an area of the body as a guide for a medical procedures. More particularly, the invention relates to applying marks to the skin of a patient undergoing radiation therapy.

### BACKGROUND OF INVENTION

It is becoming increasingly desirable to provide novel devices and methods for marking skin with marks or tattoos a patient's skin for medical procedures by means that lower the risk of transferring infections or diseases between patients and/or health care workers.

Although the use of skin marking compositions to delineate boundaries of body area requiring medical therapy or monitoring, e.g., in conjunction with radiation treatment for cancer, to mark zones for dermatological surgery, to identify areas contacted with allergens, to monitor the status of skin infections and other dermatological purposes, and to identify individual, e.g., one who have paid admission to an event, is conventional. Typically, visible compounds, such as bright purple or other brightly colored dyes or inks have been used See, for example U.S. Pat. No. 3,551,554. Such dyes and inks are adequate for marking the skin of humans for short periods of time, e.g., a few days, these visible dyes are generally not sterile for skin marking as would be needed for medical purposes, which increases the risk of transferring infections or diseases between patients and/or health care workers.

Markings, also often referred to as tattoos, are typically applied to the skin of a patient in preparation for radiation therapy or other medical treatment, as a means of clearly delineating the location of treatment set-up points. Traditionally, the marking is done by applying a drop of marking agent to the surface of the patient's skin and then tattoo the skin with a marking tip to allow the marking agent to flow on the surface of the skin. The size of the marking tip used can vary widely, some technicians preferring to work with a larger marking tips, others with a smaller tip. When marking the skin in this way, it is critical that the marking agent, such as India ink, as well as the marking tip be sterile. Although the procedure is sterile, patients may encounter side effects or allergic reactions.

Sarath et al. (U.S. Pat. No. 4,798,582; 1989) discloses a needle cartridge that is threadably mounted onto the drive shaft of an electrical tattooing instrument. Chasan (U.S. Pat. No. 5,496,304; 1996) discloses a surgical marking pen for drawing lines on the skin of a patient. The Chasan device has a handle and a fluid reservoir that communicates with a pinwheel that penetrates the skin. Arranged around the circumference of the pinwheel is a plurality of needle tips that puncture the skin, leaving a tattooed line in the epidermis of the skin.

The disadvantages of these devices are that they require special equipment or special needles. The device disclosed in Chasan has the further disadvantage of not being suited for placing individual circular or triangular markings on the skin, that is, it is a device that draws lines, rather than marks points.

U.S. Pat. Nos. 4,572,831 and 4,610,806 describe the use of skin-marking compositions that contain a furanone, which reacts with the skin in about 15-30 minutes after the application thereof to form a reaction product that is invisible under ordinary invisible light but which intensely fluoresce under ultraviolet light. This reaction product resists removal by washing from the marked area for the normal lifetime of the epidermis.

Although functionally the skin marking device of these patents perform very satisfactorily, as commercial products they have a number of deficiencies. First, the half life at ambient temperature of a solution of a fluorogenically-effective amount of fluorescamine or other dyes in a dermatologically acceptable liquid is relative short, varying from days to weeks depending on the nature of the liquid, which is unacceptable for most commercial uses. Moreover, its stability is, unfortunately, the shortest in the dermatologically most desirable liquids, such as ethanol. Second, because an acceptable solvent for most dyes typically comprises a volatile organic solvent, the solvent tends to evaporate before the solution is used up, which at best alters the concentration of the dye therein and at worst causes the solution to dry up entirely, thus reducing the number of times the marking device can be used. Third, a multiple use skin marking device which allows the tip thereof to contact the skin or other tissue of a plurality of patients can create the risk of the spread of the HIV virus or another pathological organism from an infected patient to other patients. Fourth, there is a tendency of persons involved in the skin marking procedure out of habit to treat the skin marking device of these patients in the same manner as a conventional writing instrument and as a result the marking device sometimes ends up in a pocket of a health care provider involved in a skin marking procedure and is thus unavailable for use on another patient by someone else in the same department. There is thus a desire and need in the art for providing a marking device which has the functional advantages of the skin-marking device of U.S. Pat. Nos. 4,572,831 and 4,610,806. Such a marking device would lack the commercial deficiencies described above. It is the object of the present invention to provide such a skin marking device.

### OBJECTS OF THE INVENTION

There is thus a desire and need in the art for providing a novel anti-bacterial, anti-viral, and anti-fungal single use skin-marking device adapted for marking, painting or tattooing an area of the body.

It is an object of this invention to provide multiple or single-use marking device adapted for applying an anti-microbial dye or ink device to mark the skin or other proteinaceous material, further providing a mark and which resists removal by washing. The markings can be permanent or basically resistant to removal by washing for around one to five days. Additional aspects and advantages of the invention will become apparent from the following detailed description, the drawings, and the appended claims.

### SUMMARY OF THE INVENTION

The present invention generally relates to a multiple-use or a single use disposable pen-like marking device adapted for manually marking the skin, or other proteinaceous surface with a mark, not penetrating the surface of the skin, which comprises (a) an elongate a hollow housing having a closed end, an open end and a manually distortable flexible sidewall; (b) a dry wick marking nib fitted in the open end of the housing, one end of which projects into and is in fluid communication with the interior of the housing and is impregnated with an amount of a skin marking dye possessing anti-microbial properties sufficient for a single use of the marking device; (c) a sealed frangible glass or plastic solvent tube fitted in the interior of the housing, which exterior dimensions relative to the interior dimension of the housing is such that the tube can be cracked or broken and the liquid contents thereof released into the interior of the housing by bending or squeezing the sidewall of the housing; and (d) a volume of a dermatogically acceptable solvent for the dye and anti-microbial solution in the sealed solvent tube sufficient when the tube is broken to dissolve said dye compound in the wick in the solvent released from the tube and saturate the wick marking nib with the resulting solution, thereby permitting the transfer of the resulting solution of said dye in the solvent to a surface to be marked by said dye.

For the above cited reasons, it is an object of the invention to provide a disposable anti-microbial skin-marking device that carries a cartridge of sterile marking agent and uses a tip to apply the marking agent on the skin surface. It is a further object of the invention to provide such a skin-marking device that will accept a standard tip in a range of tip dimensions.

The object of the invention is achieved by providing a skin-marking device that has a flexible housing provided with a quick-coupling type of coupler that is adapted to receive a marking tip provided with a base that mates with the quick-coupler on the housing. In the Preferred Embodiment, the quick-coupler and the housing has a female coupler and the tip base a male coupler. Any one of a range of tips provided with a quick coupler type base can be inserted into the tip end of the skin-marking device. Alternative embodiments may include other types of standard coupling mechanisms that tips are typically provided with when supplied to healthcare facilities. Thus, tips in a range of marking tips are available to the technician preparing the patient for the radiation therapy and he or she can choose the preferred tip size. The device is supplied packaged in sterile, protective material.

Accordingly, it is an important aspect of the invention to provide a marking device comprising the flexible housing containing a sealed rupturable cartridge that contains sterile marking agent, such as India-ink. The cartridge is ruptured by flexing or compressing the housing, while the tip sheath is still intact and in place. The marking agent flows into the tip and, after removing the tip cover, which is anti-fungal and mildrew-resistant, the marking device tip is used to tattoo or paint the skin of the patient with the marking agent. The cartridge contains sufficient marking agent to apply multiple tattoos to a single patient. The entire device including sterile tip is discarded after use.

The skin-marking device is provided as a sealed, sterile unit that includes the housing, the cartridge with marking agent, an anti-microbial, anti-mold and anti-mildew seal cap at the end of the housing, a quick coupler tip for receiving a tip, and a quick-coupler cap to seal the tip end of the skin-marking device before use. When ready to apply the tattoo to the patient, without penetrating the surface of the skin, the quick-coupler cap is removed, a tip with a quick-coupler base is fitted into the quick-coupler tip of the device, and the housing flexed to rupture the sealed cartridge with the marking agent. The marking agent flows into the tip, and the technician can now remove the tip sheath and apply the marking agent on the skin to precisely mark the location for treatment setup.

In a first alternative embodiment, the quick-coupler on the housing of the skin-marking device is provided with a tip that is short enough that a tip with a base that mates with the quick-coupler may be fitted over the short tip. This is an advantage in that the skin-marking devices may be provided with a typical tip for skin-marking, but a technician who has a preference for a longer or wider tip may attach the preferred tip to the skin-marking device without having to remove the short tip.

In a further alternative embodiment of the present invention, the skin-marking device can be supplied as a disposable, ready-to-use unit that includes a flexible housing with a cartridge of sterile skin-marking agent, without a quick-coupler, and already fitted with a standard gauge tip for skin-marking. The cap or cover of the marking device also contains a cap which is specially treated to be resistant to microbials, mold and mildew.

Additional aspects and advantages of the invention will become apparent from the following detailed description, the drawings, and the appended claims.

### BRIEF DESCRIPTION OF THE DRAWINGS

The foregoing features, as well as other features, will become apparent with reference to the description and figures below, in which like numerals represent like elements, and in which:

FIG. 1 shows the skin-marking device of the present invention as it is provided to the healthcare facility that applies the marking agent in accordance with the present invention;

FIG. 2 shows the skin-marking device being readied for an application of the marking agent to the skin of a patient in accordance with the present invention;

FIG. 3 shows a first alternative embodiment of the skin-marking device having a short marking tip, over which a standard marking tip can be attached in accordance with the present invention;

FIG. 4 shows a second alternative embodiment of the skin-marking device according to the present invention having a housing without a quick-coupler, and a fixed skin marking tip in accordance with the present invention.

### DETAILED DESCRIPTION OF THE INVENTION

The present invention generally relates to an anti-microbial growth-inhibiting, skin-marking device for applying a marking agent to the skin of a patient who is to undergo a medical procedure such as radiation therapy and the like.

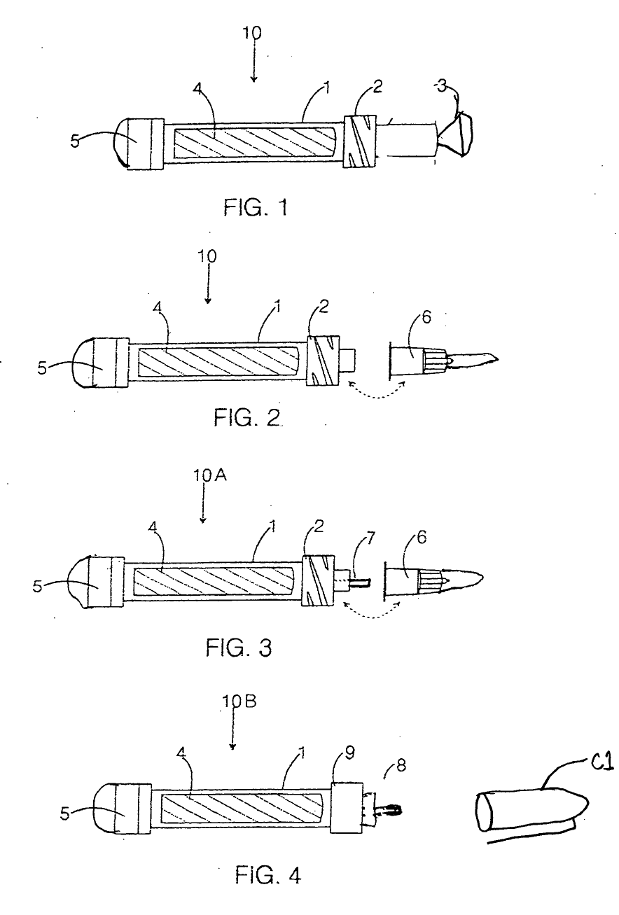

To assist in understanding the present invention, an illustrated embodiment of the present invention is presented, as well as some terms that may generally be defined. The preferred embodiment of the marking device of this invention consists of four parts or elements shown separately in FIGS. 1-3, which, when assembled as shown in FIG. 4, form a marking device 10 suitable for marking skin or proteinaceous surface with an anti-microbial dye solution. The housing, solvent tube, special cap resistant to mold and mildew and nib elements are the same structurally as those of a commercially available marking device. The marking device of this invention differs therefrom by the chemical employed as the marking compound and by being impregnated in the nib element rather than dissolved in the carrier liquid in the solvent tube.

FIG. 1 shows the preferred embodiment of a skin-marking device 10 according to the present invention. The skin-marking device 10 comprises a flexible housing 1, a quick coupler, a seal anti-microbial cap 3, a sealed cartridge 4 containing sterile marking agent, such as India-ink, and an end seal cap 5. In the Preferred Embodiment, the quick coupler seal cap 3 and the end seal cap 5 are plastic caps that snap onto the respective ends of the housing 1 to effectively seal the cartridge in the housing 1. The quick coupler 2 is adapted to receive a skin surface marking tip, which is not a needle (shown in FIG. 2) or other device that has a quick coupler base that mates with the quick coupler 2.

FIG. 2 shows the Preferred Embodiment of the skin-marking device 10 being readied for an application of marking agent. As can be seen, the quick coupler seal cap 3 has been removed from the skin-marking device 10. For purposes of illustration, the marking tip 6 with a quick coupler base has already been removed from a protective, sterile-seal sheath (not shown) and is ready to be inserted into the quick coupler 2 of the skin-marking device 10.

The technicians who apply the skin markings to the patients may have a preferences for a marking- tip size, which are not needles. Typically, the particular treatment facility will stock marking tips 6 having a quick coupler base in a range of sizes, to be used for a variety of different applications. The particular technician applying the skin-marking selects his or her preferred tip size, fits the tip 6, which is not a needle, onto the skin-marking device 10, flexes or compresses the flexible housing 1 while the tip sheath is still in place to rupture the sealed cartridge containing the marking agent, and then removes the sheath to apply the marking agent to the desired location on the skin of the patient. The marking tip 6 is a conventional tip that is typically supplied to health care facilities, enclosed in a rigid, sterile protective sheath that has a seal that is broken before use. Since the marking tip 6 and its sheath is a conventional device, well-known in the field, and does not constitute the claimed novelty of the present invention, the tip is not shown in any detail in the FIGURES and the marking tip sheath is not shown at all.

FIG. 3 shows a skin-marking device 10A that is a first alternative embodiment of the skin marking device 10 according to the present invention. The same reference designations are used to identify components of the device 10A that are identical to the components of the skin-marking device 10. As can be seen, a short marking tip 7 is provided in the quick coupler 2 of the flexible housing 1. A tip 6 that has a quick coupler base that mates with the quick coupler 2 can be inserted into the quick coupler 2, without first removing the short tip 7.

FIG. 4 shows a skin-marking device 10B that is a second alternative embodiment of the skin-marking device 10 of the present invention. Again, the same reference designations are used to identify components of the device 10B that are identical to components of the skin-marking device 10. In this second alternative embodiment, the skin-marking device 10B is provided as a ready-to-use, disposable skin-marking device that does not have a quick-coupler, but a fixed marking tip base 9, and is provided with a tip 8 that is a standard gauge for marking skin in preparation for treatment.

Preferred are solvents which are commonly employed for other dermatological or dental purposes, e.g., acetone, dissolved in an appropriate carrier fluid such as ethanol, DMF or DMSO, mineral spirits and mixtures thereof, both anhydrous and aqueous. Additionally, aqueous based solutions may be used which may aid in the dissolving of commonly used dyes. There are a large number of formulations for antibacterial cleansing solutions which include the quaternary ammonium compound Benzalkonium Chloride. At least some of the benzalkonium chloride formulations also make use of DMDM Hydantoin (e.g. “Glydant XL 1000”) as a preservative in the solution (see for example, U.S. Pat. No. 5,914,300, granted Jun. 22, 1999 in the names of Fujiwara et al., which is assigned to Lever Brothers Company).

While the invention has been described in conjunction with specific embodiments, it is evident that many alternatives, modifications and variations will be apparent to those skilled in the art in light of the foregoing description. Accordingly, the present invention attempts to embrace all such alternatives, modifications and variations that fall within the spirit and scope of the appended claims.

What is claimed is:

1. A pen-like marking device adapted for manually marking the skin or other proteinaceous surface with a mark, which comprises (a) an elongate a hollow housing having a closed end, an open end and a manually distortable flexible sidewall; (b) a dry wick marking tip fitted in the open end of the housing, one end of which projects into and is in fluid communication with the interior of the housing and is impregnated with an amount an anti-microbial solution comprising anti-bacterial, anti-fungal, and anti-viral properties sufficient for a single use of the marking device; (c) a sealed frangible glass or plastic solvent tube fitted in the interior of the housing, whose exterior dimension relative to the interior dimension of the housing is such that the tube can be cracked or broken and the liquid contents thereof released into the interior of the housing by bending or squeezing the sidewall of the housing; and (d) a volume of a dermatologically acceptable solvent for the anti-microbial solution in the sealed solvent tube sufficient when the tube is broken to dissolve the anti-microbial solution in the wick in the solvent released from the tube and saturate the wick marking nib with the resulting solution, thereby permitting the transfer of the resulting solution of the anti-microbial solution in the solvent to a surface of the marking device tip.

2. The marking device of claim 1, wherein the solvent in the tube is ethanol.

3. The marking device of claim 1, wherein the housing is formed of flexible polyvinyl chloride.

4. The marking device of claim 1, wherein the tube is glass.

5. The marking device of claim 1, wherein the nib is formed of high density polyethylene.

6. A skin-marking device for applying a marking agent on a surface layer of the skin, wherein said device is usable with a conventional, quick-coupling marking tip used in healthcare facilities for a variety of applications, said skin-marking tip having a male quick-coupler base for attaching said skin-marking tip to a conventional skin-marking device comprising:
a flexible housing that is connectable to a single skin-marking tip, fitted in the open end of the housing, one end of which projects into and is in fluid communication with the interior of the housing and is impregnated with an amount an anti-microbial solution comprising anti-bacterial, anti-fungal, and anti-viral properties sufficient for a single use of the marking device; said housing having a first end and a second end, wherein said first end is provided with a female quick-coupler for mating with said male quick-coupler base of said skin-marking tip; and
a cartridge of marking agent;
wherein said cartridge is insertable within said housing and wherein said first end and said second end each have a seal means for providing an effective seal of said housing to maintain anti-microbial activity of said marking agent;
further comprising a sealed frangible glass or plastic solvent tube fitted in the interior of the housing, whose exterior dimension relative to the interior dimension of the housing is such that the tube can be cracked or broken and the liquid contents thereof released into the interior of the housing by bending or squeezing the sidewall of the housing; and a volume of a dermatologically acceptable solvent for the anti-microbial solution in the sealed solvent tube sufficient when the tube is broken to dissolve the anti-microbial solution in the wick in the solvent released from the tube and saturate the wick marking nib with the resulting solution, thereby permitting the transfer of the resulting solution of the anti-microbial solution in the solvent to a surface of the marking device tip.

7. The device according to claim 6, wherein said seal means includes a removably attachable seal anti-microbial, anti-mold and anti-mildew cap.

8. The device according to claim 7, wherein said removably attachable seal cap is a quick-coupling cap that covers said first end and is easily removable.

9. The device according to claim 6, wherein said cartridge is a rupturable cartridge that is ruptured by flexing said housing.

10. The skin-marking device according to claim 6, further including a single short marking tip that is an integral part of said first end.

11. The skin-marking device of claim 10, wherein said skin-marking tip is coupleable with said female quick-coupler on said first end, without first removing said short marking tip from said skin-marking device.

12. A method of applying marking agent on the surface of the skin of a patient, using the skin-marking device of claim 6, said method comprising the steps of:
a. providing a set of marking tips of various sizes, each of said marking tips being said skin-marking tips having said conventional quick-coupler base;
b. providing said skin-marking device of claim 6;
c. removing said seal means from said first end;
d. selecting a tip of appropriate size from said set of marking tips;
e. inserting said quick-coupler base of said tip into said female quick-coupler;
f. flexing said housing of said device so as to rupture said cartridge; and
g. removing a protective tip sheath from said tip and marking the skin surface of a patient with said tip.

13. The device according to claim 6, wherein said device is discarded after use on a single patient and said seal means on said second end provides a permanent seal.
